(19) United States
(12) Patent Application Publication
(10) Pub. No.: US 2017/0189709 A1
(43) Pub. Date: Jul. 6, 2017

(54) TRANSCRANIAL MAGNETIC LESIONING OF THE NERVOUS SYSTEM FOR RELIEF OF INTRACTABLE PAIN

(71) Applicant: Joel Steven Goldberg, Hillsborough, NC (US)

(72) Inventor: Joel Steven Goldberg, Hillsborough, NC (US)

(21) Appl. No.: 15/463,205

(22) Filed: Mar. 20, 2017

(51) Int. Cl. A61N 2/00; A61N 2/02

(52) U.S. Cl. CPC A61N 2/008; A61N 2/006; A61N 2/02

(57) ABSTRACT

Intractable pain is a horrific cause of world-wide suffering. Nervous system excitation is a hallmark of intractable pain and lesioning of excited pathways and structures can produce sustained analgesia. This invention shows that red blood cells exposed to changing magnetic fields are disrupted, releasing hemoglobin, and that this effect is related to the dose (conformation, frequency, strength and duration) of the changing magnetic fields. Extrapolating these finding to the nervous system, transcranial magnetic lesioning of select areas of the central nervous system, in particular the anterior cingulate cortex or anterior cingulate cortices, can provide relief for patients who suffer devastating intractable pain. The changing magnetic fields are produced by electrical current through a Helmholtz coil with a soft iron core. Focusing the magnetic fields within a Helmholtz coil has advantages over focusing radiation or ultrasound because the brain can be stimulated before lesioning and the intensity of the magnetic fields can be changed according to the gap distance between the coil. In addition magnetic lesioning of tissues other than that within the nervous system may possibly provide “bloodless” surgery without exposure to radiation.

### CROSS-REFERENCES TO RELATED APPLICATIONS

None

### FEDERALLY FUNDED RESEARCH

Not applicable

### BACKGROUND OF THE INVENTION

Although medical science has made great advances in the diagnosis and treatment of pain, intractable pain, defined as pain that is refractory to all conventional therapies and causes continuous distress and suffering, is a world-wide source of morbidity and mortality. The incidence of intractable pain has been estimated to be 1 out of 1000 patients who are treated in a chronic pain clinic. (Tennant, 2007) Intractable pain is associated with chronic elevation of heart rate, blood pressure and plasma cortisol and can cause such stresses on the cardiovascular and endocrine systems that it can lead to death. (Tennant, 2007) Conditions such as central pain, arachnoiditis, complex regional pain syndrome, phantom pain and degenerative spine conditions continue to inflict horrific intractable suffering.

Present therapies to manage chronic pain include medications, injections, stimulation, surgery, and behavior modification however, intractable pain is refractory to these therapies.

Attempts to define a specific locus within the brain uniquely responsible for intractable pain have not been successful. Imaging and electrophysiologic data support the concept that pain is encoded in the brain in numerous pathways within many structures. Unlike the visual system that can be traced through discrete neural tracts and in which lesions of the system often produce consistent outcome, the sensory system for pain is diffuse. Furthermore pain is often considered to consist of both a sensory and an affective component.

At the present time there are no biomarkers in the brain that can with a high degree of sensitivity and specificity be considered a chronic pain center. The somatosensory cortices (S1 and S2) are implicated in intensity and discriminatory aspects of pain. Pure sensory strokes of the somatosensory cortices are rare. (Kim, 2007) In one patient a lesion of the post central gyrus (S1) produced contralateral sensory deficits including loss of position sense, stereognosis, and loss of two point discrimination but there was no mention in the case report of analgesia. (Derouesne, Mas, Bolgert, & Castaigne, 1984) Another patient who suffered a lesion in the post central gyrus developed contralateral decrease in pinprick, light touch, temperature and vibration with slight dysesthesia of the hand. (Shintani, Tsuruoka, & Shiigai, 2000)

Somatotopic stimulation of the motor cortex has been shown to ameliorate some forms of chronic pain. It is believed that fibers from the motor cortex are inhibitory and project to the somatosensory cortex. (Nguyen et al., 1999)

In addition to the somatosensory and motor cortices the anterior cingulate cortex (ACC) or anterior cingulate cortices have been shown to be intimately involved in pain processing. (Rainville, Duncan, Price, Carrier, & Bushnell, 1997) Cingulotomy has been performed with success in the treatment of intractable cancer pain, reflex sympathetic dystrophy, neuropathic pain, and low back pain. (Fuchs, Peng, Boyette-Davis, & Uhelski, 2014) Cingulotomy has an advantage over other lesion modalities because this procedure can be effective for diffuse intractable pain conditions.

Previous experience has shown that it is possible to stimulate neurons of the brain with transcranial magnetic stimulation (TMS). This procedure has been performed safely on thousands of patients for depression with the most serious rare side effect of seizure. (Wassermann, 1998) In order to perform the procedure a changing magnetic field of approximately 1-2 tesla is applied to the scalp and an induced electric current is produced in the brain. The changing magnetic field can be focused but usually with loss of intensity. (Peterchev et al., 2012)

A Helmholtz coil which is two solenoids arranged on a single axis has been designed for TMS in an attempt to minimize the rate of decay of the induced electric field and stimulate deep brain structures. (Williams, 2012) In this invention use of a tapered soft iron core increases the focus of the magnetic field within a Helmholtz coil.

TMS for the control of pain has been attempted over the human secondary somatosensory cortices, dorsal frontal cortex, motor cortex and ACC. However the evidence that TMS or repetitive TMS at any frequency is clinically useful for control of pain is very low. (O'Connell, Wand, Marston, Spencer, & Desouza, 2011)

There are established methods to lesion the brain without surgery. These methods, which require radiation exposure, utilize lesioning with gamma rays, x rays and positron radiation. These technologies have significant side effects including death and permanent brain damage. (Chin, Lazio, Biggins, & Amin, 2000) Ultrasound lesioning of the brain is in the developmental stage. (Fry) In this invention transcranial magnetic lesioning has advantages over other methods to lesion the brain because the tissues can be initially stimulated to confirm position and then subsequently lesioned without exposure to radiation.

This invention has the potential to help a small subset of patients with intractable pain who have failed most treatment modalities. In this invention, lesioning of the ACC with transcranial magnetic energy can produce analgesia for patients who suffer horrific intractable pain. Some of the technical aspects of transcranial magnetic lesioning include, strength, frequency, duration and focusing of the induced electric field.

The conductivity of tissue is related to the frequency of stimulation. As the frequency rises the conductivity rises and there is an inflection point in many tissues around 1,000 kilohertz. (FIGS. 1 and 2) The induced electric field is directly related to conductivity of the tissues.

The literature reporting the effects of changing magnetic fields on tissue viability is inconclusive. 60 Hz sinusoidal magnetic fields have been shown to produce apoptosis in prostate cancer cells but have no effects on mouse cerebellar tissue. (Koh et al., 2008; Mansourian, Marateb, & Vaseghi, 2016; McNamee et al., 2002)

### DESCRIPTION OF THE DRAWINGS

FIG. 1 shows the change in electrical conductivity of brain white matter as a function of the frequency of electromagnetic radiation. (Gabriel, Lau, & Gabriel, 1996)

FIG. 2 shows the change in electrical conductivity of brain grey matter as a function of the frequency of electromagnetic radiation. (Gabriel et al., 1996)

FIG. 3 shows the uniformity of the magnetic fields within a Helmholtz coil.

FIG. 4 shows the strength of the magnetic field produced by a Helmholtz coil as a function of the gap distance and radii of the coils. The y axis is the strength of the magnetic field. The x axis is the gap size. Label 1 shows the peak magnetic field when the gap distance is 0.5× radius of a coil. Label 2 shows the magnetic field when the gap distance is 1.0× radius of a coil. Label 3 shows the magnetic field when the gap distance is 1.5× radius of a coil.

FIG. 5, Label 1 shows the location of the ACC.

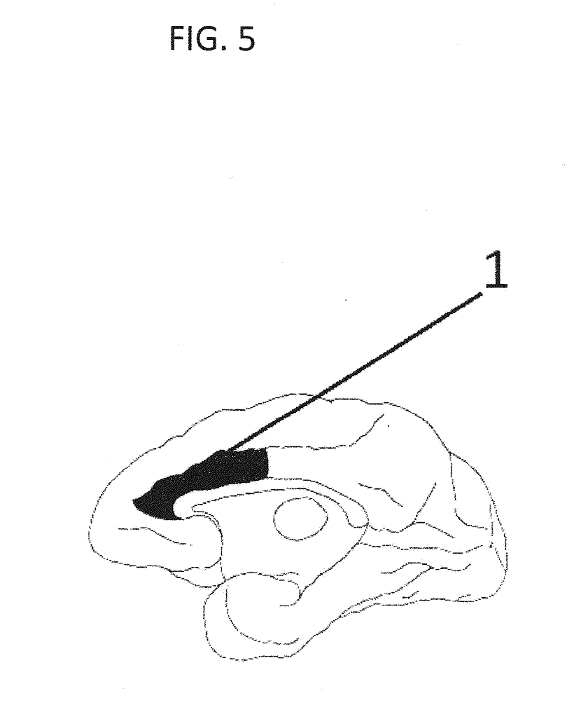

FIG. 6 shows an approximate position of a Helmholtz coil for neurolysis of the ACC. Label 1 is the coil. Label 2 is the soft iron core.

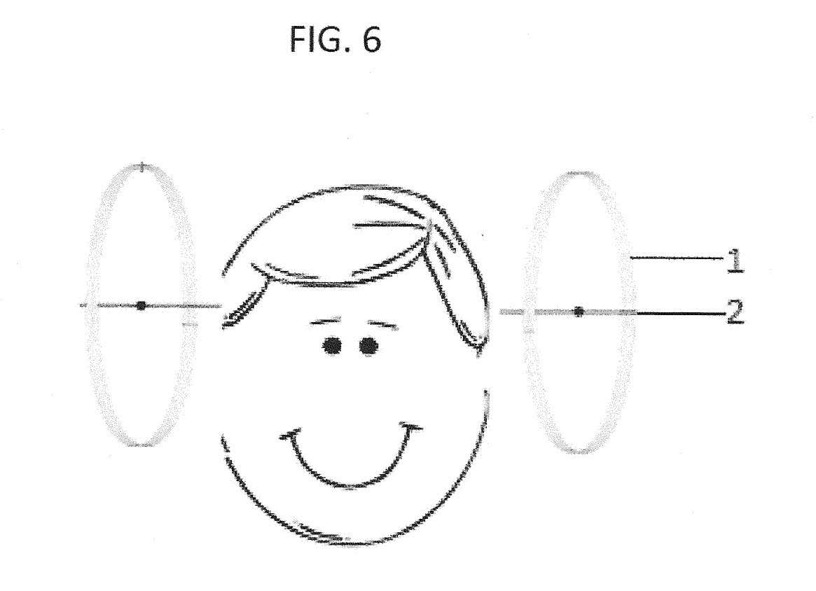

### DETAILED DESCRIPTION OF THE INVENTION

Although numerous neural pathways, structures and neurotransmitters contribute to the experience of intractable pain, there are some generally agreed upon fundamental observations:

Proposition #1: Intractable pain is an excitatory process.

A. Lesioning of the spinothalamic tract as performed in cervical cordotomy reliably produces contralateral analgesia.

B. Stimulation of inhibitory posterior column tracts of the spinal cord produces analgesia and lesions of the posterior columns produce pain.

C. Stimulation of inhibitory neurons of the motor cortex that project to the somatosensory cortex produces analgesia. (Lima & Fregni, 2008)

D. Surgical interruption of an excitatory midline dorsal column visceral pain pathway produces analgesia. (Goldberg, 2013)

E. Medications such as local anesthetics that block sodium conduction, anticonvulsants that block sodium, potassium and calcium conduction, GABAergic agonists that inhibit neural transmission and opioids that bind to G coupled receptors all have net inhibitory effects on neural transmission.

F. PET scans using cerebral blood flow correlate pain with hyperactivity as measured by increased blood flow of intracranial structures.

Proposition #2: Focal lesioning of excitatory nociceptive tracts and structures can produce sustained analgesia.

A. Lesioning excitatory nociceptive tracts is an aggressive therapy that can be effective for those patients who suffer intractable pain. These therapies include cordotomy, rhizotomy, dorsal root entry zone (DREZ) lesioning and lesioning of visceral afferent fibers of the celiac plexus, hypogastric plexus, splanchnic nerves and lumbar and stellate sympathetic ganglion.

B. Lesioning of the anterior cingulate cortex has produced analgesia for patients suffering from intractable cancer pain, complex regional pain syndrome, thoracic pain, neuropathic pain and low back pain. (Fuchs et al., 2014)

Proposition #3: Transcranial magnetic lesioning of neural structures is technically possible.

A. Previous experience has shown that it is possible to stimulate neurons of the brain with TMS. This procedure has been performed safely on thousands of patients for depression with the most serious rare side effect of seizure. (Wassermann, 1998) In order to perform the procedure a magnetic field of approximately 1-2 tesla is applied to the scalp and an induced electric field is produced in the brain. TMS can be focused but usually with loss of intensity. This invention is a not obvious extension of the TMS effects on the brain to include selected neurolysis of the brain from a changing transcranial magnetic field.

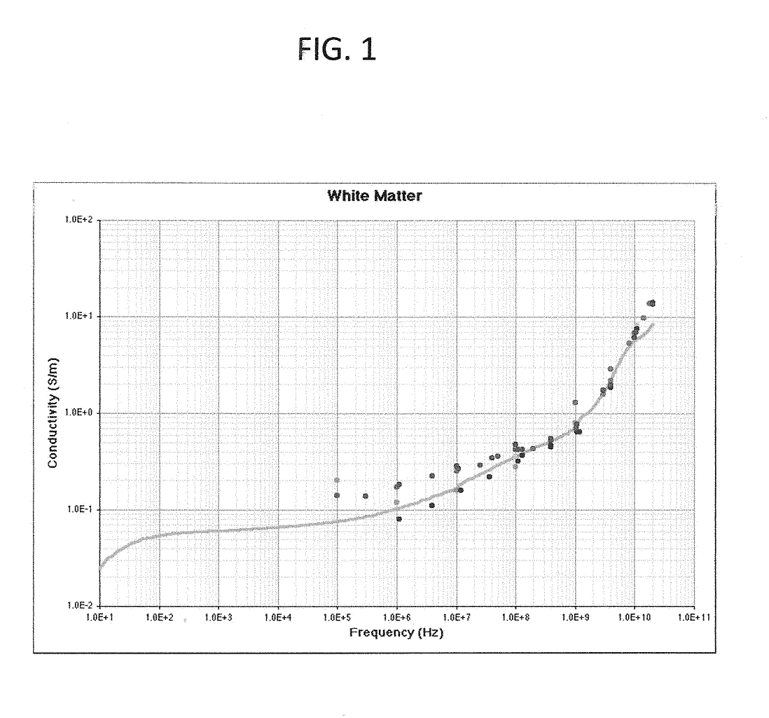

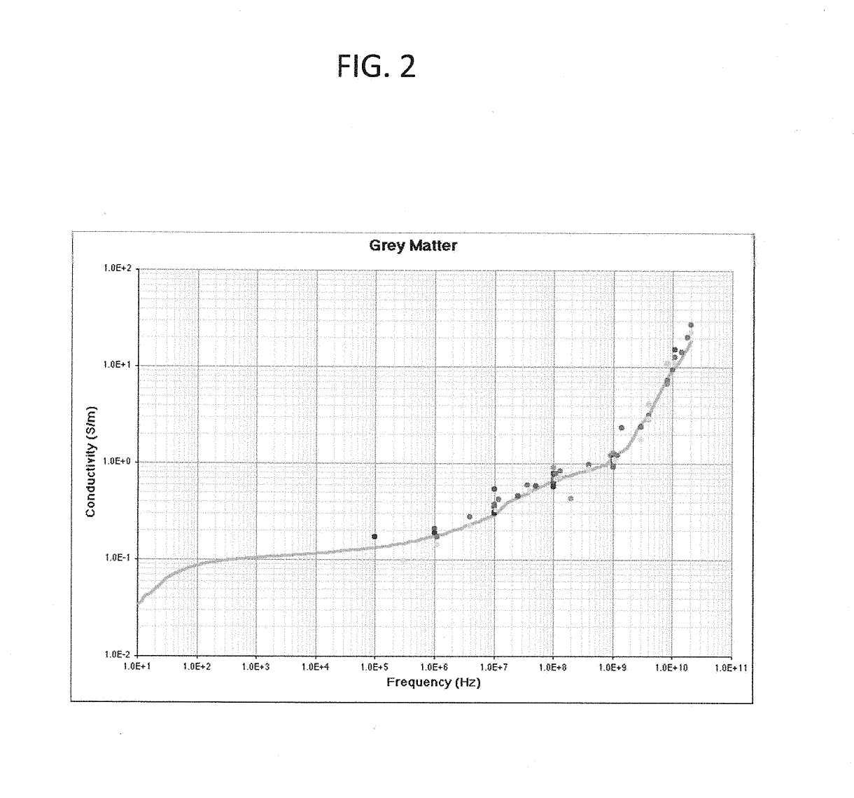

B. Increasing frequency of stimulation increases the conductivity of tissues. (FIGS. 1 and 2)

C. In this invention it was shown that changing magnetic fields can disrupt the integrity of red blood cell membranes by releasing hemoglobin, and that this effect is related to frequency of the magnetic field, strength of the magnetic field and duration of the magnetic field. (Table 2) The hemoglobin released from the disrupted red blood cells exposed to a changing magnetic field can be measured in a spectrophotometer. (Robles, Chowdhury, & Wax, 2010) This discovery can be reasonably extrapolated to destruction of membranes of neural structures and other tissues.

D. The predicted magnetic field strength for brain lesioning based on electromagnetic field strength require to disrupt red blood cell membranes is 0.02 tesla to 1.5 tesla with a square wave conformation. A magnetic field strength of 1.5 tesla approaches the maximum obtainable field strength without special cooling of the magnetic coil and without saturation of the soft iron core.

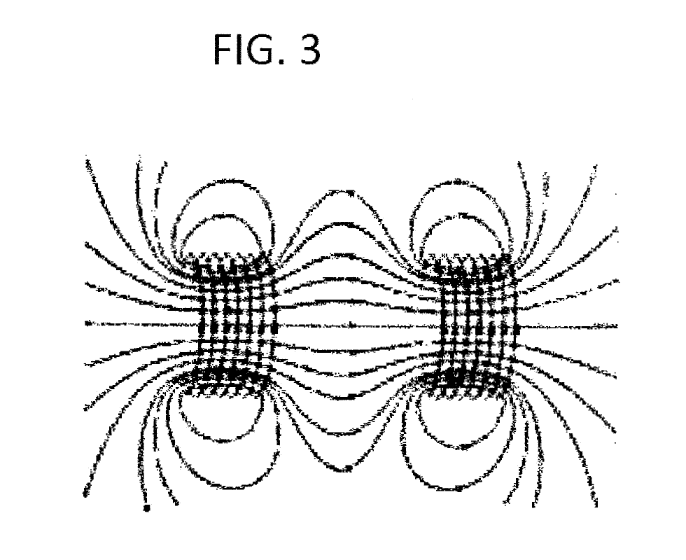

E. The conformation of the changing magnetic field would maintain the characteristics of a Helmholtz coil. (FIG. 3) The formula for the B field at the mid-plane of the gap between the coils with an air core is:

B=·π·N·I/5.5^{1/2}·a·10⁻⁷ tesla

B=magnetic field at the mid-plane
I=current
N=number of turns per coil
a=the radius of the coils and separation between the coils

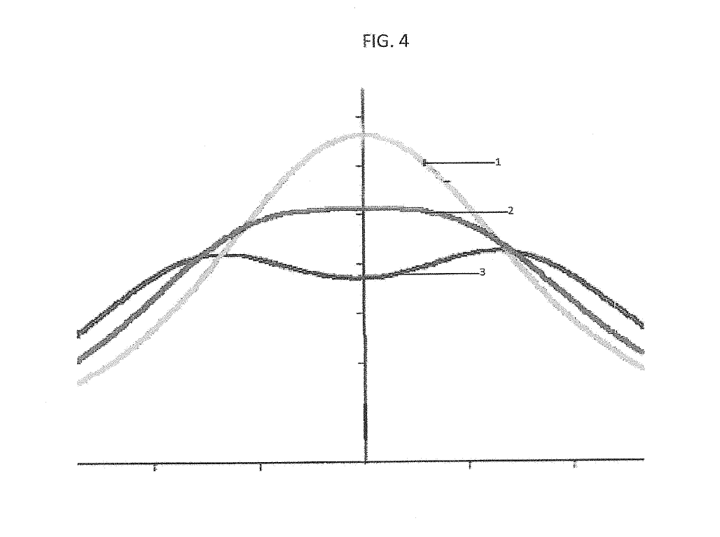

As the gap between the coils decreases the magnetic field at the mid-plane increases. (FIG. 4) This property of the Helmholtz coil can be used to focus the magnetic field. Unlike radiation and ultrasound technologies in which the energy dissipates from the source to the desired location the magnetic flux density of a Helmholtz coil is such that the B field increases with distance from the poles for a gap distances that are at least larger than ½ of the coil radius. It is predicted therefore that unwanted lesioning of tissue in the path of the magnetic field would be less likely with magnetic lesioning than with radiation or ultrasound technologies.

F. Identification of the focal point within ACC is aided with MRI imaging and with patient reports of stimulation before lesioning.

G. The predicted frequency of the changing magnetic field based on the frequency require to disrupt red cell membranes and conductivity of brain tissue is 100 kilohertz to 1,000 kilohertz.

H. The predicted duration of the brain tissue exposed to a changing magnetic field based on the duration to disrupt red cell membranes is 10-120 minutes. The shortest duration possible would permit the lesioning to occur without movement.

I. The predicted pathway of the magnetic field would be skin and subcutaneous tissue, temporalis muscle, frontal bone, lateral and medial portion of the anterior cerebral cortex to the ACC or anterior cingulate cortices. The line of this field would not disturb the temporal lobe or major sensory and motor tracts.

Proposition #4: Even though specific brain biomarkers of pain have not been elucidated, lesion(s) most likely to produce analgesia can be deduced by considering the human experiment data of 1) autoradiography of mu receptors in the brain, 2) PET scans of location of brain mu receptors and 3) cerebral blood flow as measured by fMRI activity during experimentally produced pain. (Table 1)

A. Focal lesions of the somatosensory cortices (S1and S2) are rare. Literature reviews indicate that these lesions produce anesthesia of the contralateral side but not analgesia. Studies suggest that S2 is more concerned with the intensity of the nociceptive stimulus rather than the location of the stimulus.

B. Motor cortex lesions do not produce analgesia but motor cortex stimulation produces analgesia suggesting that connections between the motor and sensory cortex are inhibitory.

C. Lesions of the ACC can produce analgesia that is not somatotopically specific. The preferred embodiment of this invention based on results from cingulotomy would be to lesion the ACC or anterior cingulate cortices.

TABLE 1
Location of significant expression of mu opioid
receptors in the brain and fMRI (cerebral
blood flow) activity during experimentally
produced pain (Chen et al., 2008; Duerden &
Albanese, 2013; Henriksen & Willoch,
2008; Jones et al., 1991)

| | Brodmann | Auto- | PET | |
|---|---|---|---|---|
| Common name | area | radiography | scan | fMRI |
| Anterior cingulate | 24, 32, 33 | + | + | + |
| cortex | | | | |
| Primary | 1, 2, 3 | − | − | − |
| somatosensory | | | | |
| cortex (S1) | | | | |
| Secondary | 40, 43 | + | + | + |
| somatosensory | | | | |
| cortex (S2) | | | | |
| Motor cortex | 40 | − | − | − |

### Benefits to Society

Intractable pain causes horrific suffering. Those suffering have minimal quality of life. This invention provides a method to decrease pain with non-invasive lesioning of the CNS without radiation. In addition it provides a method to perform focal non-invasive lesioning of tissues beyond that of the central nervous system and potential “bloodless” surgery.

### Experimental Section

### Methods

A solution of 50 ml of phosphate buffered saline, 1000 units of porcine heparin and 0.5 mg of dextrose was the standard nutrient solution. 10 microliters of blood were obtained from a finger stick and mixed with 20 microliters of nutrient solution in a micro centrifuge tube. 500 microliters of phosphate buffered saline (PBS) was added to each micro centrifuge tube. The suspended cells were exposed to changing magnetic fields of variable frequencies, durations and strengths within a Helmholtz coil. The micro centrifuge tubes were insulated and temperature measurements within the coil showed no appreciable changes compared to the ambient temperature. This observation excluded temperature effects on red blood cell viability.

The Helmholtz coil consisted of two solenoids each with 480 turns of wire with a radius of 2.5 cm and ½ inch soft iron core. The field is nearly uniform at a distance 1 radius from the inner pole. The frequencies of the magnetic field were produce by an arbitrary wave form generator (OWAN Model AG 1012F, Fujian, China) and amplified by (Model TS 250-1 function generator amplifier, Accel Instruments, Irvine, Calif.). Additionally the amplifier's output was monitored with an oscilloscope and a gauss meter measured the magnetic field.

After exposure to the changing magnetic fields the red blood cells and controls were centrifuged at 1500 rpm for 5 minutes to produce a red blood cell pellet and supernatant. 400 microliters of supernatant with PBS were assayed in a spectrophotometer (Ultrospec III, Pharmacia, Cambridge, England) at 541 nm for hemoglobin absorption. (Kahn, Watkins, & Bermes, 1981) The 541 wavelength was determined by best fit Beer's law plot at a peak absorbance. This corresponded to reported absorbance for oxy-hemoglobin and it was felt that conversion to cyano-hemoglobin was not needed for the assay. Realizing that the differences in hemoglobin absorbance may be very small between the experimental sample exposed to a changing magnetic field and controls, sample quartz cuvettes were perfectly matched to absorbance values of 3 decimal places between each experiment and the spectrophotometer was recently calibrated.

### Results

An increase in hemoglobin absorption (Abs) was observed in the sample group exposed to the changing magnetic field compare to the control samples. (Table 2) n=22, p<0.00001.

TABLE 2
Effects of changing magnetic fields of various frequencies, durations
and strengths on red cell membrane integrity as measured by
spectrophotometric absorbance at 541 nm of free hemoglobin.

| Time | | | | | Am- | B |
|---|---|---|---|---|---|---|
| (min- | Frequency | Abs | Abs | | perage | Field |
| utes) | (Hz) | (sample) | (control) | Δ Abs | (initial) | (μ tesla) |
| 10 | 1 | 0.060 | 0.058 | 0.002 | 3.0 | 4455 |
| 10 | 10 | 0.056 | 0.020 | 0.036 | 3.0 | 4524 |
| 10 | 100 | 0.065 | 0.020 | 0.045 | 3.0 | 4517 |
| 30 | 100 | 0.084 | 0.058 | 0.026 | 3.0 | 4517 |
| 30 | 100 | 0.040 | 0.005 | 0.035 | 3.0 | 4517 |
| 30 | 100 | 0.095 | 0.083 | 0.012 | 3.0 | 4517 |
| 10 | 1,000 | 0.060 | 0.039 | 0.021 | 3.0 | 4514 |
| 10 | 10,000 | 0.081 | 0.076 | 0.005 | 3.0 | 4514 |
| 10 | 100,000 | 0.082 | 0.050 | 0.032 | 3.0 | 4503 |
| 10 | 100,000 | 0.067 | 0.068 | −0.001 | 3.0 | 4503 |
| 10 | 100,000 | 0.045 | 0.048 | −0.003 | 3.0 | 4503 |
| 10 | 100,000 | 0.077 | 0.079 | −0.002 | 3.0 | 4503 |
| 30 | 100,000 | 0.070 | 0.039 | 0.031 | 3.0 | 4503 |
| 30 | 100,000 | 0.074 | 0.036 | 0.038 | 3.0 | 4503 |
| 60 | 100,000 | 0.173 | 0.119 | 0.054 | 3.0 | 4503 |
| 60 | 100,000 | 0.055 | 0.022 | 0.033 | 3.0 | 4530 |
| 120 | 200,000 | 0.013 | 0.001 | 0.012 | 2.0 | 2096 |
| 120 | 200,000 | 0.024 | 0.005 | 0.019 | 2.0 | 2096 |
| 10 | 1,000,000 | 0.040 | 0.030 | 0.010 | 2.0 | 3672 |
| 30 | 1,000,000 | 0.074 | 0.036 | 0.038 | 3.0 | 4459 |
| 30 | 1,000,000 | 0.087 | 0.061 | 0.026 | 2.5 | 3786 |
| 120 | 1,000,000 | 0.049 | 0.039 | 0.010 | 1.0 | 1059 |

n = 22, p < 0.00001

### Discussion

Even with magnetic field strengths as low as 2000 μ tesla, there was evidence of hemolysis caused by exposure of red blood cells to a changing magnetic field. It is predicated that higher magnetic field strengths will be required to permanently disrupt the membranes of neural tissue. Unlike neurons and supporting neural structures, red blood cells do not contain DNA so the effects of the changing magnetic field on nuclear integrity could not be assessed.

### REFERENCES

Tennant F. The Intractable Pain Patient's Handbook for Survival. Glenview, Ill., USA: Pain Treatment Topics; 2007.

Kim J S. Patterns of sensory abnormality in cortical stroke: evidence for a dichotomized sensory system. Neurology. Jan. 16, 2007; 68(3):174-180.

Derouesne C, Mas J L, Bolgert F, Castaigne P. Pure sensory stroke caused by a small cortical infarct in the middle cerebral artery territory. Stroke. July-August 1984; 15(4):660-662.

Shintani S, Tsuruoka S, Shiigai T. Pure sensory stroke caused by a cerebral hemorrhage: clinical-radiologic correlations in seven patients. AJNR. American journal of neuroradiology. March 2000; 21(3):515-520.

Nguyen J P, Lefaucheur J P, Decq P, et al. Chronic motor cortex stimulation in the treatment of central and neuropathic pain. Correlations between clinical, electrophysiological and anatomical data. Pain. September 1999; 82(3):245-251.

Rainville P, Duncan G H, Price D D, Carrier B, Bushnell M C. Pain affect encoded in human anterior cingulate but not somatosensory cortex. Science. Aug.15 1997; 277(5328):968-971.

Fuchs P N, Peng Y B, Boyette-Davis J A, Uhelski M L. The anterior cingulate cortex and pain processing. Frontiers in integrative neuroscience. 2014; 8:35.

Wassermann E M. Risk and safety of repetitive transcranial magnetic stimulation: report and suggested guidelines from the International Workshop on the Safety of Repetitive Transcranial Magnetic Stimulation, Jun. 5-7, 1996. Electroencephalography and clinical neurophysiology. January 1998; 108(1):1-16.

Peterchev A V, Wagner T A, Miranda P C, et al. Fundamentals of transcranial electric and magnetic stimulation dose: definition, selection, and reporting practices. Brain stimulation. October 2012; 5(4):435-453.

Williams P I, Marketos, P., Crowther, L. J., Jiles, D. C. New Designs for Deep Brain Transcranial Magnetic Stimulation. IEEE Transactions on Magnetics. 2012; 48(3):1171-1178.

O'Connell N E, Wand B M, Marston L, Spencer S, Desouza L H. Non-invasive brain stimulation techniques for chronic pain. A report of a Cochrane systematic review and meta-analysis. European journal of physical and rehabilitation medicine. June 2011; 47(2):309-326.

Chin L S, Lazio B E, Biggins T, Amin P. Acute complications following gamma knife radiosurgery are rare. Surgical neurology. May 2000; 53(5):498-502; discussion 502.

Fry F J, Sanghvi. N. T., Inventor. ULTRASOUND BRAIN LESIONING SYSTEM. U.S. Pat. No. 4,951,6531990.

McNamee J P, Bellier P V, McLean J R, Marro L, Gajda G B, Thansandote A. DNA damage and apoptosis in the immature mouse cerebellum after acute exposure to a 1 mT, 60 Hz magnetic field. Mutation research. Jan. 15, 2002; 513(1-2):121-133.

Mansourian M, Marateb H R, Vaseghi G. The effect of extremely low-frequency magnetic field (50-60 Hz) exposure on spontaneous apoptosis: The results of a meta-analysis. Advanced biomedical research. 2016; 5:141.

Koh E K, Ryu B K, Jeong D Y, Bang I S, Nam M H, Chae K S. A 60-Hz sinusoidal magnetic field induces apoptosis of prostate cancer cells through reactive oxygen species. International journal of radiation biology. November 2008; 84(11):945-955.

Gabriel S, Lau R W, Gabriel C. The dielectric properties of biological tissues: II. Measurements in the frequency range 10 Hz to 20 GHz. Physics in medicine and biology. November 1996; 41(11):2251-2269.

Lima M C, Fregni F. Motor cortex stimulation for chronic pain: systematic review and meta-analysis of the literature. Neurology. Jun. 10, 2008; 70(24):2329-2337.

Goldberg J S. Chronic opioid therapy and opioid tolerance: a new hypothesis. Pain research and treatment. 2013; 2013:407504.

Robles F E, Chowdhury S, Wax A. Assessing hemoglobin concentration using spectroscopic optical coherence tomography for feasibility of tissue diagnostics. Biomedical optics express. Jul. 27, 2010; 1(1):310-317.

Henriksen G, Willoch F. Imaging of opioid receptors in the central nervous system. Brain: a journal of neurology. May 2008; 131(Pt 5):1171-1196.

Chen T L, Babiloni C, Ferretti A, et al. Human secondary somatosensory cortex is involved in the processing of somatosensory rare stimuli: an fMRI study. Neuroimage. May 1, 2008; 40(4):1765-1771.

Duerden E G, Albanese M C. Localization of pain-related brain activation: a meta-analysis of neuroimaging data. Human brain mapping. January 2013; 34(1):109-149.

Jones A K, Qi L Y, Fujirawa T, et al. In vivo distribution of opioid receptors in man in relation to the cortical projections of the medial and lateral pain systems measured with positron emission tomography. Neuroscience letters. May 13, 1991; 126(1):25-28.

Kahn S E, Watkins B F, Bermes E W, Jr. An evaluation of a spectrophotometric scanning technique for measurement of plasma hemoglobin. Annals of clinical and laboratory science. March-April 1981; 11(2):126-131.

Having described my invention, I claim:

1. A method to control intractable pain by transcranial magnetic lesioning of the anterior cingulate cortex or anterior cingulate cortices of a human comprising:
a) a Helmholtz coil with a soft iron core
b) a changing magnetic field generated by a square wave with a frequency of 10 hertz to 1,000 kilohertz with a strength of 0.2 tesla to 1.4 tesla for a duration of 10 minutes to 120 minutes.

2. A method to produce lesions in human tissues located within a Helmholtz coil by exposure to said tissues of a changing magnetic field comprised of:
a) a frequency range of 10 hertz to 1,000 kilohertz
b) a magnetic flux density between 0.2 tesla to 1.4 tesla and
c) a duration of 10 minutes to 120 minutes.

3. The method of claim 2 where the lesioned tissues are located within the central nervous system.

4. The method of claim 2 where the lesioned tissues are located outside the central nervous system.
